(19) United States
(12) Patent Application Publication
(10) Pub. No.: US 2010/0190735 A1
(43) Pub. Date: Jul. 29, 2010

(54) Mouthwash and Method of Using Same for the Treatment of Mucositis or Stomatitis

(75) Inventor: Deepak BHASIN, Richmond Hill (CA)

Correspondence Address: Adorno & Yoss, LLC, 1349 West Peachtree Street, NE, Suite 1500, Atlanta, GA, 30309, US

(73) Assignee: MYREX PHARMACEUTICALS INC., Toronto (CA)

(21) Appl. No.: 12/752,721

(22) Filed: Apr. 1, 2010

Related U.S. Application Data
(63) Continuation-in-part of application No. PCT/CA2007/001743, filed on Oct. 3, 2007.
Application No. PCT/CA2007/001743 is a continuation-in-part of application No. 11/692,737, filed on Mar. 28, 2007, now abandoned.
(60) Provisional application No. 60/786,376, filed on Mar. 28, 2006.

(51) Int. Cl. A61K 31/7048; A61K 31/56; A61P 29/00; A61P 31/04

(52) U.S. Cl. 514/31; 514/171

(57) ABSTRACT

An oral rinse formulation for the treatment or prophylaxis of mucositis or stomatitis comprises one or more of the following components in combination with pharmaceutically acceptable excipients or additives: a) at least one corticosteroid; b) at least one anti-histamine; c) at least one topical anaesthetic; and, d) at least one anti-fungal antibiotic agent. A stable nystatin containing formulation is also provided comprising a buffering agent, a preservative, a chelating agent and an anti-oxidant.

### CROSS REFERENCE TO PRIOR APPLICATIONS

This application is a Continuation in Part of PCT application number PCT/CA2007/001743, filed on Oct. 3, 2007, which is a Continuation in Part of U.S. application Ser. No. 11/692,737, filed on Mar. 28, 2007 (now abandoned), which claims priority from U.S. provisional application No. 60/786,376, filed on Mar. 28, 2006. The entire contents of all the aforementioned applications are incorporated herein by reference.

### BACKGROUND OF THE INVENTION

The present invention relates to oral hygiene and in particular, to mouthwashes for the treatment and prophylaxis of mucositis and stomatitis of the oral cavity and to a method of using such a mouthwash.

Mouthwashes are typically used as part of an oral hygiene regime which may also include brushing and flossing to maintain a satisfactory level of oral hygiene. Such mouthwashes are generally classified as cosmetic, therapeutic or a combination of the two. Cosmetic rinses are commercial over-the-counter products that help remove oral debris before or after brushing, temporarily suppress bad breath, diminish bacteria in the mouth and refresh the mouth with a pleasant taste. Therapeutic rinses often have the benefits of their cosmetic counterpart, but also contain an added active ingredient, for example chlorhexidine that helps protect against some oral diseases such as gingivitis. Typically, such mouthwashes are alcohol-based.

However, available mouthwashes are unsuitable for hospital patients presenting with oral complications as a result of, for example, infection or other lesions as a consequence of chemotherapy or radiotherapy for the treatment of neoplastic disease. Such oral complications may include mucositis or stomatitis. Mucositis or stomatitis presents in approximately 40% of patients with neoplastic disease.

Examples of chemotherapeutic drugs that frequently cause mucositis and/or stomatitis include alkylating agents, for example melphalan and busulphan; antimetabolites, for example, cytarabine, floxuridine, 5-fluorouracil, mercaptopurine, methotrexate, and thioguanine and cytotoxic drugs, for example, bleomycin, actinomycin D, daunorubicin, cisplatin, etoposide, mytomycin, vinblastine and vincristine.

The terms mucositis and stomatitis are often used interchangeably but may include some general distinctions. Mucositis describes a toxic inflammatory reaction affecting the gastro-intestinal tract, which may result from exposure to chemotherapeutic agents or ionizing radiation. Mucositis typically manifests as an erythematous burn-like lesion, or as random focal-to-diffuse lesions. Stomatitis refers to an inflammatory reaction affecting the oral mucosa, with or without ulceration, that may be caused or intensified by pharmacological, particularly chemotherapeutic treatments or by radiotherapy. Stomatitis can range from mild to severe; a patient presenting with the severe stomatitis is unable to take anything by mouth.

Current oral cleaning care in such patients may include gently cleaning the mouth, moisturizing the lips and mouth, and relieving pain and swelling. A soft toothbrush cleans teeth well and gently. Cleansing agents can include “salt and soda” (half a teaspoon of salt and two tablespoons of sodium bicarbonate in 32 ounces of warm water), saline, sterile water or sodium bicarbonate (one teaspoon in 8 ounces of water). Hydrogen peroxide diluted in equal amounts of water or weak salt water can be used in some cases. Mostly, physicians have resorted to these rather limited, temporary relief options.

It is therefore extremely important that mucositis and stomatitis be prevented whenever possible or at the very least that they be reduced in their severity and possible complications. Currently there are a number of intervening therapies to choose from. For example, The Joanna Briggs Institute of The Royal Adelaide Hospital has provided some guidance on the treatment of mucositis. However, there is no high quality synthesis of research evidence for these intervention therapies (www.joannabriggs.edu.au/best_practice/bp5.php, 19 Feb. 2004).

Of particular interest to the current application are the variety of mouthwashes previously used having mixed actions against mucositis and stomatitis. Such mouthwashes typically include benzydamine hydrochloride, corticosteroids and chamomile (www.joannabriggs.edu.au/best_practise/bp5.php).

Formulations such as those disclosed in U.S. Pat. No. 5,635,489; U.S. Pat. No. 4,961,926 and U.S. Pat. No. 5,102,870 describe the use of growth factors and stimulation factors such as granulocyte-macrophage colony stimulating factor and granulocyte-colony stimulating factor.

Further attempts at treatment and or prophylaxis of mucositis and stomatitis include the use of nucleoside derivatives which can be formulated into lozenges or mouthwashes and the like to coat the oral cavity or other mucosal areas such as disclosed in U.S. Patent Publication No. 2003/0236217 A1.

Other formulations include tetracycline as disclosed in U.S. Pat. No. 6,946,118; hyaluronic acid, glycyrrhetinic acid and polyvinylpyrrolidone in mixture with excipients and adjuvants as disclosed in U.S. Pat. No. 6,828,308; the use of an anti-microbial peptide, preferably protegrin applied topically which has broad spectrum anti-microbial activity, good stability and adheres well to all mucosa as disclosed in U.S. Pat. No. 6,025,326; the use of glutamine as disclosed in U.S. Pat. No. 5,545,668 and the use of triclosan as disclosed in U.S. Pat. No. 5,945,089

Despite the widespread recognition that mucositis is a serious problem, no effective treatment currently exists and, more often than not, any level of patient care is typically palliative. In addition to the lack of effective treatments for mucositis, physicians are unsure of the exact mechanism by which the ulcerations occur in mucositis. It is known that the initial exposure to a chemotherapeutic agent causes a release of cytokines from the epithelial tissues. Subsequently, mitosis is disturbed in the epithelia. Finally, there are alterations in the bacterial flora of the oral cavity. It is unknown which of these occurrences, if any, is responsible for the mucosal damage. Despite the fact that significant quantities or inflammatory mediators are released by the epithelium, conventional anti-inflammatory agents have been unsuccessful in human efficacy studies of the disease.

Some of the known and effective treatments for mucositis involves the use of the antibiotics nystatin and amphotericin B. These drugs have been used for the preventive and curative treatment of fungal infections such as oral candidiasis and, in particular, for cancer patients. Due to a lack of commercially available anti-fungal mouthwashes, oral or mouth rinse formulations containing these antifungal agents are commonly prepared in a clinical setting for immediate use. One possible reason for the lack of nystatin and/or amphotericin B mouth rinse formulations is that such formulations are relatively unstable and, therefore, must be used within a short period of time. For example, in one study of mixtures of nystatin and amphotericin B, in combination with sodium bicarbonate, it was determined that the maximum amount of time that such formulations can remain stable is roughly 3 to 4 days (J. Groeschke, et al.; Stability of Amphotericin B and Nystatin in Antifungal Mouthrinses Containing Sodium Hydrogen Carbonate, J. Pharm. and Biomed. Anal., v. 42, 3 (2006), pp. 362-366).

Thus, the available mouthwashes are effective at treating only one aspect of mucositis and other oral complications and not treating the complete range of symptoms that present in patients. Further, the effective antifungal mouth rinses have a very limited stability. There exists a need, therefore, for a stable formulation for treating a variety of symptoms and causes of mucositis.

### SUMMARY OF THE INVENTION

In one aspect, the present invention provides an oral rinse formulation comprising one or more of the following components in combination with pharmaceutically acceptable excipients or additives:

a) at least one corticosteroid;

b) at least one anti-histamine;

c) at least one topical anaesthetic;

d) at least one anti-fungal antibiotic agent.

In another aspect, the formulation can include an antibiotic agent.

In a further aspect, the invention provides a method of treating or preventing mucositis or stomatitis comprising applying to a patient the above mentioned oral rinse formulation.

In another aspect, the invention provides a stable, anti-fungal oral rinse formulation comprising:
an anti-fungal agent;
a phosphate buffer;
a preservative;
a chelating agent; and,
an antioxidant.

In one embodiment, the anti-fungal agent is chosen from nystatin and amphotericin B. In a further embodiment, the above formulation comprises: at least one corticosteroid; at least one antihistamine; and at least one topical anaesthetic.

### DETAILED DESCRIPTION OF THE INVENTION

Accordingly, the present invention provides, in one embodiment, a mouthwash comprising one or more of the following active ingredients:
at least one corticosteroid;
at least one anti-histamine;
at least one topical anaesthetic;
at least one anti-fungal antibiotic agent.

Optionally, the formulation may include an antibiotic agent as well.

In another embodiment, the invention provides a mouthwash formulation comprising an anti-fungal agent, a phosphate buffer, a preservative, a chelating agent and an antioxidant. This formulation preferably also includes at least one corticosteroid, at least one antihistamine, and at least one topical anaesthetic. More preferably, the anti-fungal agent of this formulation comprises nystatin or amphotericin B and, most preferably, nystatin.

Preferably, the mouthwash in accordance with the present invention is used in the treatment and/or prophylaxis of oral conditions such as mucositis and/or stomatitis.

Although the mouthwash (or mouth rinse) of the present invention includes various components that are known in the art, the inventors note that there is no commercially available formulation that combines the subject components to provide an effective, single formulation that addresses a large variety of symptoms associated with mucositis and/or stomatitis. This is also clearly indicated in the recent article by J. Groeschke, et al. Further, in addition to providing such multipurpose formulation, the inventors have also found an unexpectedly high stability of the formulation, particularly when an anti-fungal is included. More specifically, the formulation described below including nystatin was found to be stable for over 2 months, and at least as long as 12 months at room temperature or under refrigeration, thereby greatly exceeding the stability expected according to the prior art, such as the article by J. Groeschke, et al.

Preferably the mouthwash in accordance with the present invention further comprises water and pharmaceutically acceptable excipients or additives such as:
one or more oils, such as an oil selected from the group comprising anethole, anisole, camphor, methyl salicylate, vanillin, eugenol, furaneol, linalool, menthol, thymol, cinnamaldehyde, citral, methyl butanoate, pentylbutanoate, pentylpentanoate, tea tree oil, peppermint oil, spearmint oil, pineapplemint oil and eucalyptus oil;
sweetening agents, for example sorbitol;
thickening agents, such as xanthan gum, carrageenan, carbomer, or HPMC (hydroxypropyl methyl cellulose);
preservative agents, such as sodium benzoate, methyl paraben, or propyl paraben;
water;
emulsifiers, such as polysorbate 80 (or Tween™ 80);
and/or at least one antacid such as aluminium or magnesium hydroxide.

It will be appreciated by persons skilled in the art that the above list of excipients and/or additives is provided merely by way of example and that various other such components may be used in the formulation of the present invention.

Oral mucositis results in specific damage that includes the shedding of the mucosal lining of the mouth (desquamation), ulceration and atrophy. Aluminum and magnesium hydroxide antacids coat and protect the oral cavity from damage and provide for re-growth of normal oral tissue.

The corticosteroid component provides anti-inflammatory action directly on the oral mucosa and works to limit the inflammatory response associated with mucositis. Preferably, the corticosteroid is dexamethasone.

In patients being treated with chemotherapy and radiation, steps may be taken to prevent bacterial infection within the oral cavity. For this reason, in an optional embodiment, the mouthwash formulation of the invention may contain an antibiotic component. Such antibiotic components may be of the macrolide type and may be selected from the group consisting of erythromycin, azithromycin, clarithromycin, dirithromycin, roxithromycin carbomycin A, josamycin, kitasamycin, oleandomycin, spiramycin, troleandomycin, tylosin, cethromycin, ansamycin and telithromycin. In one aspect the antibiotic is erythromycin. Once ulcerations develop inside the mouth, local oral bacteria colonize the wound and release cell wall products into the mucosa, resulting in an amplification of a tissue destructive cycle. The antibiotic component, i.e. Erythromycin, limits such bacterial colonization. As will be understood, limiting the extent of bacterial infection would promote healing of the affected tissues.

Alternatively, the antibiotic may be any of the following, alone or in combination:
an aminoglycoside, for example, amikacin, gentamicin, kanamycin, neomycin, netilmicin, streptomycin, and tobramycin;
a carbacephem, for example, loracarbef;
a carbapenem, for example, ertapenem, imipenem/cilastatin, and meropenem;
a cephalosporin (first generation), for example, cefadroxil, cefazolin, cephalexin;
a cephalosporin (second generation), for example, cefaclor, cefamandole, cefoxitin, cefprozil, and cefuroxime;
a cephalosporin (third generation), for example, cefixime, cefdinir, cefditoren, cefoperazone, cefotaxime, cefpodoxime, ceftazidime, ceftibuten, ceftizoxime, and ceftriaxone;
a cephalosporin (fourth generation), for example, cefepime;
a glycopeptide, for example, teicoplanin, vancomycin;
a penicillin, for example, amoxicillin, ampicillin, azlocillin, carbenicillin, cloxacillin, dicloxacillin, flucloxacillin, mezlocillin, nafcillin, penicillin, piperacillin and ticarcillin;
a polypeptide, for example, bacitracin, colistin, and polymyxin B;
a quinolone, for example, ciprofloxacin, enoxacin, gatifloxacin, levofloxacin, lomefloxacin, moxifloxacin, norfloxacin, ofloxacin, and trovafloxacin;
a sulfonamide, for example, mafenide, prontosil, sulfacetamide, sulfamethizole, sulfanilimide, sulfasalazine, sulfisoxazole, trimethoprim, and trimethoprim-sulfamethoxazole;
a tetracycline, for example, demeclocycline, doxycycline, minocycline, and oxytetracycline, tetracycline, and
another antibiotic, for example, chloramphenicol, clindamycin, ethambutol, fosfomycin, furazolidone, isoniazid, linezolid, metronidazole, nitrofurantoin, pyrazinamide, quinupristin/dalfopristin, rifampin, and spectinomycin.

The anti-fungal antibiotic agent may be of the polyene type and may be selected from the group consisting of nystatin, amphotericin B and natamycin. In one aspect, the anti-fungal agent is nystatin or amphotericin B.

Patients being treated with chemotherapy and radiation are immuno-compromised. This leads to not only increased risk of bacterial infection but also to an increased risk of fungal infection, thus advancing the risk and degree of oral mucositis. Nystatin and amphotericin B act to prevent and limit the degree of fungal infection.

Alternatively, the anti-fungal compound may be selected from an imidazole, for example, miconazole, ketoconazole, clotrimazole, econazole, mebendazole, bifonazole, butoconazole, fenticonazole, isoconazole, oxiconazole, sertaconazole, sulconazole, thiabendazole or tiaconazole; a triazole, for example, fluconazole, itraconazole, ravuconazole, posaconazole or voriconazole; an allylamine, for example, terbenafine, amorolfine, naftifine or butenafine or an echinocandin such as caspofungin or micafungin or any combinations thereof.

The topical anaesthetic may be selected from benzocaine, mepivacaine, ropivacaine, bupivacaine, lidocaine, prilocalne, procaine, cloroprocaine or tetracaine. In one aspect the topical anaesthetic is a combination of lidocaine and tetracaine.

Pain associated with oral mucositis can be extremely debilitating and lead to poor oral food intake. In extreme cases patients may require feeding tubes if the ulceration continues to advance. Tetracaine and lidocaine act locally to provide relief from pain.

Preferably, the anti-histamine may be selected from the group comprising a first generation H1 receptor antagonist, a second generation H1 receptor antagonist, a third generation H1 receptor antagonist, a H2 receptor antagonist, a H3 receptor antagonist and a H4 receptor antagonist.

Diphenyhdramine, an antihistamine, helps to sooth soreness, burning, itching (urticaria and pruritis) and inflammation, symptoms that are normally associate with mucositis.

The first generation H1 receptor antagonist may be selected from an ethylenediamine, for example, mepyramine or antazoline; an ethanolamine, for example, diphenhydramine, carbinoxamine, doxylamine, clemastine or dimenhydrinate; an alkylamine, for example, pheniramine, chlorphenamine, dexchlorphenamine, brompheniramine or triprolidine; a piperazine, for example, cyclizine, hydroxyzine, meclizine or a tricyclic, for example, promethazine, alimemazine, cyproheptadine or azatadine. In one aspect, the anti-histamine is diphenhydramine.

The second generation H1 receptor antagonist may be azelastine, levocabastine or olopatadine.

The H3 receptor antagonist may be thioperamide, clobenpropit or impromidine.

The H4 receptor antagonist may be thioperamide.

Preferably, dexamethasone is used in the range of from 0.005 mg/ml to 0.025 mg/ml, or, more preferably, narrower ranges such as 0.01 mg/ml to 0.02 mg/ml or 0.015 mg/ml to 0.02 mg/ml. In one preferred embodiment, dexamethasone is present in a concentration of 0.016 mg/ml.

Preferably, diphenhydramine is used in a range of from 1 mg/ml to 2 mg/ml or, more preferably, in narrower ranges such as 1 mg/ml to 1.5 mg/ml, 1.2 mg/ml to 1.4 mg/ml, or 1.25 mg/ml to 1.3 mg/ml. In one preferred embodiment, dexamethasone is present in a concentration of 1.67 mg/ml.

Preferably, lidocaine is used in a range of from 1 mg/ml to 20 mg/ml (i.e. 0.1% to 2%) although narrower ranges may also be used. In one preferred embodiment, lidocaine is present in a concentration of 10 mg/ml.

Preferably, nystatin is used in an amount greater than 10,000 iu/ml. More preferably, Nystatin is used in a range of 10,000 iu/ml to 50,000 iu/ml, or 20,000 iu/ml to 40,000 iu/ml, or 30,000 iu/ml to 35,000 iu/ml, or most preferably at a concentration of 33,333 iu/ml.

When included in the formulation of the invention, erythromycin is used in a range of from 5 mg/ml to 15 mg/ml, or, more preferably, in narrower ranges such as 10 mg/ml to 15 mg/ml, 12 mg/ml to 15 mg/ml, or 12.5 mg/ml to 15 mg/ml.

In a further embodiment, the present invention provides a mouthwash comprising 0.015 mg/ml dexamethasone, 1.25 mg/ml diphenhydramine, 0.5% lidocaine, 20000 iu/ml nystatin, polysorbate 80, spearmint oil, sorbitol, xanthan gum, sodium benzoate, purified water, aluminium hydroxide and magnesium hydroxide. Optionally, the formulation may also include 12.5 mg/ml erythromycin.

In a further aspect the present invention provides a method of treating or preventing oral conditions such as mucositis or stomatitis comprising rinsing the oral cavity with the mouthwash of the invention.

The present invention also provides, in a further embodiment, a kit for the treatment or prophylaxis of mucositis or stomatitis comprising a container having an amount of mouthwash in accordance with the present invention together with a set of instructions for using the mouthwash. The kit may optionally provide a cup or other container for conveniently dispensing an amount of the mouthwash from the container. Advantageously the cup may also have markings or other indicators for the convenient dispense of a measurement of a therapeutically effective dose of the mouthwash.

In a further embodiment, the invention provides a mouthwash or mouth rinse formulation that is exhibits long term stability. The stable formulation of the invention includes nystatin as the anti-fungal component. In a preferred embodiment, the nystatin containing formulation includes a buffering agent, a preservative, a chelating agent and an anti-oxidant.

In another aspect, the invention provides a stable, anti-fungal oral rinse formulation comprising: an anti-fungal agent (chosen from nystatin, amphotericin B and mixtures thereof); a phosphate buffer; a preservative (such as methyl paraben and/or propyl paraben); a chelating agent (such as EDTA); and an antioxidant (such as citric acid or a salt thereof).

In a further aspect of the invention, the above formulation also includes at least one corticosteroid; at least one antihistamine; and at least one topical anaesthetic.

### EXAMPLES

The following examples are provided to illustrate the invention and are not intended to limit the scope of the invention in any way.

Introduction

As discussed above, there are currently no mouth rinses available on the market containing Nystatin, Dexamethasone sodium phosphate, Lidocaine HCL and Diphenhydramine HCl available. Moreover, there are no commercially available mouth rinses containing nystatin and this is believed to be mainly due to the poor stability characteristics of formulations containing this drug. For this reason, various mouth rinse (or mouthwash) formulations were prepared to study the stability characteristics. The aim of this project was to develop a stable formulation containing at least nystatin and, preferably the various other treatment agents listed above. As discussed further below, a prototype formulation was developed and its stability was tested at room temperature. The product was found to be stable at room temperature for over three months, and at least as long as 12 months. The product was also found to remain stable for at least 12 months at 4° C.

Materials and Methods

Table 1 below summarises the formulations studied. As shown, nine sample preparations were examined, identified as samples B1 to B9. Where the samples differed in formulation, a notation is included in Table 1.

The following procedure was typical of that followed in preparing the formulations of the example. As indicated above, the specific concentrations of the various components for the trials is indicated Table 1.

Step 1. Preparation of 1.2% HPMC (hydroxypropyl methyl cellulose) solution for the final volume
1.1) Heat 70 mL DI water to 90° C.
1.2) Slowly add 2.4 g HPMC with continuous stirring and heating at 90° C. for 1 hour.
1.3) Cool down to room temperature with continuous stirring (about 1 hour).
1.4) Add 130 mL cold DI (de-ionized) water with continuous stirring for 60 minutes.
1.5) Set aside for nystatin suspension preparation in step 3.

Step 2. Preparation of water soluble API's (active pharmaceutical ingredients) and other ingredients
2.1) Dissolve 0.60 g methyl paraben (0.2%) and 60 mg propyl paraben (0.02%) in 15.0 g propylene glycol (5%) and stir at 40° C. for 10 min or until completely dissolved.
2.2) Add 3.0 g Tween™ 80 (1.0%) then stir for 10 min at 40° C.
2.3) Add 150 mL DI water and cool down to room temperature.
2.4) Add each of the following remaining ingredients and all soluble API's with continuous stirring until completely dissolved. No heating.
   i. 1.5 g citric acid
   ii. 4.3018 g sodium phosphate dibasic
   iii. 1.5 g sucralose
   iv. 6.444 mg dexamethasone 21-phosphate disodium salt
   v. 581.61 mg diphenhydramine HCl
   vi. 3.741 g lidocaine HCl

Step 3. Preparation of Nystatin Suspension
3.1) Slowly add 1.6431 g nystatin to 120 mL 1.2% HPMC solution (from step 1) with continuous stirring. The final HPMC concentration in the formulation will therefore be 0.5%.
3.2) Add 0.60 g EDTA (0.2%) and mix well.

Step 4. Mix solution from step 2 and nystatin suspension from step 3. Add 1.2 mL NFB mixed berry flavor. Mix well.

Step 5. Measure pH. If it is not 6.5±0.5, adjust to pH 6.5±0.5 with 100 mM phosphate buffer (Na₂HPO₄).

Step 6. Adjust final volume to 300 mL with water. Mix well and verify pH again.

At the end of Step 6, the formulation (300 ml) will have the composition as listed in the following table. Items marked as * comprise the active drug ingredients. Items marked as ** comprise the alternative components used for a placebo formulation.

| | | Freebase | | | Weight of | |
|---|---|---|---|---|---|---|
| | | Concn | | | component | |
| Note | Chemical Name | (mg/mL) | Concentration | Units | added | |
| | Propylene Glycol | | 5.0 | % | 15.0 | g |
| | Methyl Paraben | | 0.20 | % | 0.6 | g |
| | Propyl Paraben | | 0.02 | % | 60 | mg |
| | Tween 80 | | 1.0 | % | 3.0 | g |
| | Citric Acid | | 0.50 | % | 1.5 | g |
| | Sodium Phosphate dibasic | | 100.0 | mM | 4.3018 | g |
| | Sucralose | | 0.50 | % | 1.5 | g |
| * | Dexamethasone 21- | 0.016 | 0.02148 | mg/mL | 6.444 | mg |
| | phosphate | | | | | |
| | disodium salt | | | | | |
| * | Diphenhydramine HCl | 1.67 | 1.9387 | mg/mL | 0.581 | g |
| * | Lidocaine HCl | 10 | 12.47 | mg/mL | 3.741 | g |
| | HPMC | | 0.5000 | % | | |
| * | Nystatin | 33,333 IU/mL | 5.477 | mg/mL | 1.6431 | |
| | EDTA acid calcium disodium | | 0.20 | % | 0.6 | g |
| | Mixed NFB Berry Flavor | | 0.40 | % | 1.2 | g |
| ** | Titanium Oxide | | 5.00 | mg/mL | 1.5 | g |
| ** | D&C Yellow No. 10 | | 0.001 | % | | |

The formulations were stored at room temperature and tested upon formulation (i.e. time=0) and at 2, 3 and 4 month time points for identity, potency, pH, preservative assay, physical appearance and color. Formulation B8 was used for testing stability and the results of this analysis at the various time points are provided in Tables 2 to 6.

Quantification of the respective ingredients was done using high performance liquid chromatography (HPLC). A difference in calculated quantity of less than 5% from the intial formulation (i.e. time=0) amount was considered a “pass”, that is, indicative of minimal degradation. The relative standard deviation for the HPLC calculations was 3%.

For the re-dispersibility study, the following procedure was followed:

manually shake the sample for 5 seconds.

observe the bottom of the glass bottle.

if settlement or clumping is found, shake for another 5 seconds and observe.

the sample is identified as re-dispersible if no settlement or clumping is found on the bottom of the glass bottle.

### CONCLUSIONS

All the samples prepared for the stability study were found to meet the desired criteria. The mouth rinse of the invention was found to be stable, when stored room temperature, for at least up to 4 months, and at least up to 12 months. As also shown in Table 6, the formulation of the invention was found to remain stable for at least 12 months when stored at 4° C., suggesting that longer storage periods would be possible at room temperatures but also under refrigeration. At “accelerated conditions”, that is, when stored at 40° C., the formulation was found to remain stable for less than 2 months. These results illustrate an unexpected stability of a nystatin containing formulation particularly when stored at room temperature or under refrigeration. The stability of the nystatin containing formulation is believed to be attributable to the presence of the buffering agent (pH control), preservatives (i.e. paraben), the chelating agent (i.e. EDTA), and the anti-oxidant (i.e. citrate). The data obtained from this study indicates that the formulation of the invention exhibits long term stability and, therefore, provides a unique, single dose formulation that addresses various symptoms associated with mucositis.

Although the invention has been described with reference to certain specific embodiments, various modifications thereof will be apparent to those skilled in the art without departing from the purpose and scope of the invention as outlined in the claims appended hereto. Any examples provided herein are included solely for the purpose of illustrating the invention and are not intended to limit the invention in any way. The disclosures of all prior art recited herein are incorporated herein by reference in their entirety.

TABLE 1
Sample formulations prepared for stability study

| | | Concentration (free base | | | |
|---|---|---|---|---|---|
| Ingredient Type | Active/Excipient | concentration) | Grade | Other Candidates | Notes |
| Corticosteroid | Dexamethasone sodium phosphate | 0.016 mg/mL | USP grade | | H₂O soluble |
| | salt | | | | |
| Anti-histamine | Diphenhydramine HCl | 1.67 mg/mL | USP grade | | H₂O soluble |
| Anesthetic | Lidocaine HCl | 10 mg/mL | USP grade | | H₂O soluble |
| Anti-fungal | Nystatin | 33,333 IU/mL (5.477 mg/mL) | USP grade | | slightly soluble- |
| | | | | | 4 mg/ml in H₂O |
| Anti-oxidant | Citric Acid | 0.5% w/v | USP | | |
| Chelating agent | Calcium disodium EDTA | 0.2% (w/v) | USP | | |
| Wetting agent | Polysorbate 80 (Tween ™ 80) | 1.0% for B1, B2, B8, B9 | USP | Polysorbate 20, | H₂O soluble |
| (emulsifier) | | 1.2% for B3, B4, B5 | | Poloxamer 407 | |
| | | 1.5% for B6 | | | |
| Sweetening agent | Sucralose | 0.5% (w/v) | USP | | H₂O soluble |
| Thickener | HPMC (Hydroxy Propyl Methyl | 0.25%(w/v) for B1, B2, B7 | USP | (Sodium Carboxyl | H₂O soluble |
| | Cellulose) | 0.3% for B3, B4 | | Methyl cellulose) | |
| | (from Dow Chemical F4M) | 0.4% for B5, B6, B8 | | SCMC | |
| | | 0.5% for B9 | | | |
| Preservative | Methyl paraben, Propyl paraben | 0.2% Methyl paraben (w/v); | USP | | parabens are |
| | | 0.02% Propyl paraben | | | slightly soluble in |
| | | B2 - no propyl paraben | | | hot water |
| Tonicity adjusting | Dextrose | used where the osmolality | | NaCl | H₂O soluble |
| factor | | of the formulation | | | |
| | | is <280 mOsm/kg | | | |
| Flavour | Sensient NFB mixed berry flavor | 0.4% v/v | Pharma. | | |
| | | | grade | | |
| Solvent | Purified water | >85% (w/w) | | | |
| Buffer agent | Sodium phosphate Dibasic | 100 mM | USP | citrate phosphate | H₂O soluble |
| | | | | buffer | |

TABLE 2
Sample characteristics upon formulation (time = 0)

| Test | Active/Excipient | Specification Limits | Method | Results | Pass/fail |
|---|---|---|---|---|---|
| Identity | Dexamethyasone sodium phosphate; | Positive | HPLC | Conforms | Pass |
| | Diphenhydramine HCl; Lidocaine HCl | | | | |
| Identity | Nystatin | Positive | HPLC | Conforms | Pass |
| Potency | Dexamethasone sodium phosphate | 90 to 110% of label claim; less than 5% change in | HPLC | 103.2% | Pass |
| | | stability sample | | | |
| Potency | Diphenhydramine HCl | 90 to 110% of label claim; less than 5% change in | HPLC | 100.6% | Pass |
| | | stability sample | | | |
| Potency | Lidocaine HCl | 90 to 110% of label claim; less than 5% change in | HPLC | 100.4% | Pass |
| | | stability sample | | | |
| Potency | Nystatin | 90 to 110% of label claim; less than 5% change in | HPLC | 108.5% | Pass |
| | | stability sample | | | |
| pH | | 6 to 7 | | 6.37 | Pass |
| Preservative assay | Methyl paraben, Propyl paraben | | HPLC | Methyl | Pass |
| | | | | paraben: 100.8% | |
| | | | | Propyl | |
| | | | | paraben: 99.1% | |
| Physical | Color | No significant change in stability sample | Visual | Light yellow color | Pass |
| Appearance | | Light yellow color | | | |
| Re-dispersibility | | No settlement or clump on the bottom of the | | Conforms | Pass |
| | | product bottle | | | |

TABLE 3
Sample characteristics after 2 months (at room temperature and at 40° C.)

| | | | | Results | | Results | |
|---|---|---|---|---|---|---|---|
| Test | Active/Excipient | Specification Limits | Method | at room Temp | Pass/fail | at 40° C. | Pass/fail |
| Identity | Dexamethyasone sodium | Positive | HPLC | Conforms | Pass | Conforms | Pass |
| | phosphate; | | | | | | |
| | Diphenhydramine | | | | | | |
| | HCl; Lidocaine HCl | | | | | | |
| Identity | Nystatin | Positive | HPLC | Conforms | Pass | Conforms | Pass |
| Potency | Dexamethasone sodium | 90 to 110% of label claim; less than | HPLC | 98.1% | Pass | 100.8% | Pass |
| | phosphate | 5% change in stability sample | | | | | |
| Potency | Diphenhydramine HCl | 90 to 110% of label claim; less than | HPLC | 100.8% | Pass | 100.2% | Pass |
| | | 5% change in stability sample | | | | | |
| Potency | Lidocaine HCl | 90 to 110% of label claim; less than | HPLC | 99.7% | Pass | 98.1% | Pass |
| | | 5% change in stability sample | | | | | |
| Potency | Nystatin | 90 to 110% of label claim; less than | HPLC | 106.8% | Pass | 98.4% | Fail |
| | | 5% change in stability sample | | | | | |
| pH | | 6 to 7 | | 6.3 | Pass | 6.34 | Pass |
| Preservative | Methyl paraben, Propyl | | HPLC | Methyl paraben: | Pass | Methyl paraben: | Pass |
| assay | paraben | | | 97.6% | | 95% | |
| | | | | Propyl paraben: | | Propyl paraben: | |
| | | | | 99.3% | | 99.1% | |
| Physical | Color | No significant change in stability | Visual | Light yellow color; no | Pass | | |
| Appearance | | sample | | significant change | | | |
| | | Light yellow color | | | | | |
| Re- | | No settlement or clump on the bottom | | Conforms | Pass | | |
| dispersibility | | of the product bottle | | | | | |

TABLE 4
Sample characteristics after 3 months (at room temperature and at 40° C.)

| | | | | Results | | Results | |
|---|---|---|---|---|---|---|---|
| Test | Active/Excipient | Specification Limits | Method | at room Temp | Pass/fail | at 40° C. | Pass/fail |
| Identity | Dexamethyasone sodium | Positive | HPLC | Conforms | Pass | Conforms | Pass |
| | phosphate; | | | | | | |
| | Diphenhydramine | | | | | | |
| | HCl; Lidocaine HCl | | | | | | |
| Identity | Nystatin | Positive | HPLC | Conforms | Pass | Conforms | Pass |
| Potency | Dexamethasone sodium | 90 to 110% of label claim; less than | HPLC | 102.7% | Pass | 97.1% | Pass |
| | phosphate | 5% change in stability sample | | | | | |
| Potency | Diphenhydramine HCl | 90 to 110% of label claim; less than | HPLC | 101.85% | Pass | 101.7% | Pass |
| | | 5% change in stability sample | | | | | |
| Potency | Lidocaine HCl | 90 to 110% of label claim; less than | HPLC | 101.8% | Pass | 102.15% | Pass |
| | | 5% change in stability sample | | | | | |
| Potency | Nystatin | 90 to 110% of label claim; less than | HPLC | 106% | Pass | 88.8% | Fail |
| | | 5% change in stability sample | | | | | |
| pH | | 6 to 7 | | 6.3 | Pass | 6.25 | Pass |
| Preservative | Methyl paraben, Propyl | | HPLC | Methyl paraben: | Pass | Methyl paraben: | Pass |
| assay | paraben | | | 100.1% | | 95.7% | |
| | | | | Propyl paraben: | | Propyl paraben: | |
| | | | | 100.4% | | 99.4% | |
| Physical | Color | No significant change in stability | Visual | Light yellow color; no | Pass | | |
| Appearance | | sample | | significant change | | | |
| | | Light yellow color | | | | | |
| Re- | | No settlement or clump on the bottom | | Conforms | Pass | | |
| dispersibility | | of the product bottle | | | | | |

TABLE 5
Sample characteristics after 4 months (at room temperature and at 40° C.)

| | | | | Results | | Results | |
|---|---|---|---|---|---|---|---|
| Test | Active/Excipient | Specification Limits | Method | at room Temp | Pass/fail | at 40° C. | Pass/fail |
| Identity | Dexamethyasone sodium | Positive | HPLC | Conforms | Pass | Conforms | Pass |
| | phosphate; Diphenhydramine | | | | | | |
| | HCl; Lidocaine HCl | | | | | | |
| Identity | Nystatin | Positive | HPLC | Conforms | Pass | Conforms | Pass |
| Potency | Dexamethasone sodium | 90 to 110% of label claim; less than | HPLC | (not quantified) | Pass | 102.7 | Pass |
| | phosphate | 5% change in stability sample | | | | | |
| Potency | Diphenhydramine HCl | 90 to 110% of label claim; less than | HPLC | (not quantified) | Pass | 100.1% | Pass |
| | | 5% change in stability sample | | | | | |
| Potency | Lidocaine HCl | 90 to 110% of label claim; less than | HPLC | (not quantified) | Pass | 102.9% | Pass |
| | | 5% change in stability sample | | | | | |
| Potency | Nystatin | 90 to 110% of label claim; less than | HPLC | 105.3% | Pass | 92.4% | Fail |
| | | 5% change in stability sample | | | | | |
| pH | | 6 to 7 | | (not quantified) | Pass | 6.27 | Pass |
| Preservative | Methyl paraben, Propyl | | HPLC | (not quantified) | Pass | Methyl paraben: | Pass |
| assay | paraben | | | | | 97.1% | |
| | | | | | | Propyl paraben: | |
| | | | | | | 99.2% | |
| Physical | Color | No significant change in stability | Visual | (not quantified) | Pass | | |
| Appearance | | sample | | | | | |
| | | Light yellow color | | | | | |
| Re- | | No settlement or clump on the bottom | | Conforms | Pass | | |
| dispersibility | | of the product bottle | | | | | |

TABLE 6
Sample characteristics after 12 months (at room temperature and at 4° C.)

| | | | | Results | | Results | |
|---|---|---|---|---|---|---|---|
| Test | Active/Excipient | Specification Limits | Method | At Room Temp | Pass/fail | at Fridge 4° C. | Pass/fail |
| Identity | Dexamethyasone sodium | Positive | HPLC | Conforms | Pass | Conforms | Pass |
| | phosphate; Diphenhydramine | | | | | | |
| | HCl; Lidocaine HCl | | | | | | |
| Identity | Nystatin | Positive | HPLC | Conforms | Pass | Conforms | Pass |
| Potency | Dexamethasone sodium | 90 to 110% of label claim; less than | HPLC | 100.1 | Pass | 96.3% | Pass |
| | phosphate | 5% change in stability sample | | | | | |
| Potency | Diphenhydramine HCl | 90 to 110% of label claim; less than | HPLC | 103.0% | Pass | 102.4% | Pass |
| | | 5% change in stability sample | | | | | |
| Potency | Lidocaine HCl | 90 to 110% of label claim; less than | HPLC | 101.4% | Pass | 101.4% | Pass |
| | | 5% change in stability sample | | | | | |
| Potency | Nystatin | 90 to 110% of label claim; less than | HPLC | 109.6% | Pass | 101.7% | Pass |
| | | 5% change in stability sample | | | | | |
| pH | | 6 to 7 | | 6.28 | Pass | 6.31 | Pass |
| Preservative | Methyl paraben, Propyl | | HPLC | Methyl P - 98.2% | Pass | 99.8% | Pass |
| assay | paraben | | | Propul P - 99.1 | | | |
| Physical | Color | No significant change in stability | Visual | Light yellow color; | Pass | | |
| Appearance | | sample | | no significant | | | |
| | | Light yellow color | | change | | | |
| Re- | | No settlement or clump on the bottom | | Conforms | Pass | | |
| dispersibility | | of the product bottle | | | | | |

We claim:

1. A stable oral rinse formulation comprising the following components in combination with pharmaceutically acceptable excipients or additives:
a) at least one corticosteroid;
b) at least one anti-histamine;
c) at least one topical anaesthetic; and
d) at least one anti-fungal antibiotic agent;
wherein, said formulation exhibits stability for at least 2 months at room temperature.

2. The formulation of claim 1 having the following composition:
0.005 to 0.025 mg/ml dexamethasone;
1 to 2 mg/ml diphenhydramine;
1 to 20 mg/ml lidocaine; and
10,000 to 50,000 iu/ml nystatin.

3. The formulation of claim 2 wherein the additives are chosen from the group consisting of: polysorbate 80, spearmint oil, sorbitol, xanthan gum, sodium benzoate, purified water, aluminium hydroxide, dextrose, sucralose, flavorings and magnesium hydroxide.

4. The formulation of claim 2 further comprising at least one buffering agent, at least one preservative, at least one chelating agent and at least one anti-oxidant.

5. The formulation of claim 4 wherein the buffering agent is a phosphate buffer, the preservative is methyl paraben or propyl paraben, the chelating agent is EDTA, and the anti-oxidant is citric acid or a salt thereof.

6. Use of the formulation of claim 1 for the treatment of oral lesions.

7. The use of claim 1 for the treatment of mucositis or stomatitis.

8. A stable, anti-fungal oral rinse formulation comprising nystatin and a buffering agent, a preservative, a chelating agent and an anti-oxidant, said formulation exhibiting stability for at least 2 months at room temperature.

9. The formulation of claim 8 wherein the buffering agent is a phosphate buffer.

10. The formulation of claim 8 wherein the preservative is methyl paraben or propyl paraben.

11. The formulation of claim 8 wherein the chelating agent is EDTA.

12. The formulation of claim 8 wherein the anti-oxidant is citric acid or a salt thereof.

13. The formulation of claim 8 wherein the nystatin is present in an amount greater than 10,000 iu/ml.

14. A stable, anti-fungal oral rinse formulation comprising:
an anti-fungal antibiotic agent selected from nystatin and amphotericin B;
a phosphate buffer;
a preservative;
a chelating agent; and,
an antioxidant;
said formulation exhibiting stability for at least 2 months at room temperature.

15. The formulation of claim 14 wherein the nystatin is present in an amount between 10,000 and 50,000 iu/ml.

16. The formulation of claim 14 wherein the preservative is chosen from methyl paraben, propyl paraben and mixtures thereof.

17. The formulation of claim 14 wherein the chelating agent is ethylenediamine tetraacetic acid (EDTA).

18. The formulation of claim 14 wherein the antioxidant is citric acid or a salt thereof.

19. The formulation of claim 14 further comprising:
at least one corticosteroid;
at least one antihistamine; and
at least one topical anaesthetic.

20. The formulation of claim 19, wherein:
the corticosteroid is dexamethasone at a concentration of between 0.005 to 0.025 mg/ml;
the antihistamine is diphenhydramine, at a concentration of between 1 to 2 mg/ml; and,
the topical anaesthetic is lidocaine, at a concentration of between 1 to 20 mg/ml.
